(19) United States
(12) Patent Application Publication
(10) Pub. No.: US 2009/0014009 A1
(43) Pub. Date: Jan. 15, 2009

(54) Toroidal endotracheal cuffs for ventilator associated pneumonia reduction

(75) Inventors: Fung-jou Chen, Appleton, WI (US); Jeffrey Dean Lindsay, Appleton, WI (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 11/827,793

(22) Filed: Jul. 13, 2007

(51) Int. Cl. A61M 16/04

(52) U.S. Cl. 128/207.15

(57) ABSTRACT

An airway ventilation device for insertion in an elongate body cavity is provided. The airway ventilation device includes an endotracheal tube and an inflation cuff. The inflation cuff is toroidal in shape and includes one or more recessed attachment zones which allow for movement of the endotracheal tube without breaking the seal between the inflation cuff and the elongate body cavity.

### BACKGROUND

Current endotracheal tubes and other airway sealing medical devices utilize an inflation cuff that presses against the sides of the trachea or other body cavity as a fluid barrier. The integrity of this cuff seal is critical, as there is a substantial amount of secretions that accumulate on top of the tubes during use that are harmful to the patient if allowed to enter the patient's lungs or pass beyond such other devices in other applications.

Traditionally, these endotracheal tubes are constructed such that the inflation cuffs are attached to the endotracheal tube at an attachment point which is longitudinally away from the inflation cuff.

Often, during a medical procedure, the medical professional will be required to manipulate the endotracheal tube in a transverse or longitudinal manner or the patient may shift resulting in movement of the tube. During manipulation or movement of the endotracheal tube, gaps often form between the tracheal walls and the inflation cuffs because the endotracheal tubes are constructed in the traditional manner with longitudinally away attachment zones and because these traditional inflation cuffs may slowly deflate over time.

These gaps are particularly disturbing because they allow for the passage or aspiration of secretions into the lungs. These secretions often contain bacteria that may cause ventilator acquired pneumonia which according to some studies accounts for 7-8% of all deaths in hospital Intensive Care Units.

Attempts have been made to prevent leakage of secretions around the inflation cuffs of endotracheal tubes into the lungs. These attempts, however, often add significant costs. For example, gelling sealants have been used to flow into and fill in gaps between the inflation cuff and the tracheal wall. Additionally, antimicrobial materials have been placed on the tube itself or around the inflation cuffs. Although antimicrobials are generally effective, a significantly lower amount of anti-microbial materials could be used with an inflation cuff having better sealing characteristics. This would result in lower manufacturing costs.

Thus, there remains a need for a more economical endotracheal tube having an inflation cuff that can maintain its seal with the trachea even while being manipulated in a transverse or longitudinal direction or during patient shifting.

### SUMMARY OF INVENTION

The present invention provides for an airway ventilation device for insertion in an elongate body cavity. The airway ventilation device includes a tube having a distal end and a proximal end. The device also includes a toroidal shaped inflation cuff comprising an outer surface, an inner surface, a distal end, a medial portion, and a proximal end. The toroidal shaped inflation cuff is adapted to attach to the tube at least at two attachment zones on the tube. The first attachment zone, in comparison to the proximal end of the inflation cuff, is desirably located at a distance further from the proximal end of the tube, and second attachment zone, in comparison to the distal end of the inflation cuff, is desirably located at a distance further from the distal end of the tube and wherein the outer surface is in communication with the trachea upon inflation.

Desirably, the inflation cuff is inflated by introducing a fluid through one or more lumens that open up into the interior of the inflation cuff.

Desirably, the outer surface of the inflation cuff will remain in substantially stationary communication with the trachea when the tube is moved in a longitudinal or transverse direction. This movement will desirably be greater than the amount of longitudinal or transverse movement allowed by the residual volume of a similarly-sized non-toroidal shaped inflation cuff when it remains in stationary communication with the trachea upon longitudinal or transverse movement.

This decreases the chance of a space opening between the outer surface of the inflation cuff and the tracheal wall upon movement of the endotracheal tube and it consequently decreases the chances of infectious secretions moving past the inflation cuff and entering into the lungs.

The device may include at least two attachment zones, which, upon inflation, are substantially aligned with the medial portion of the inflation cuff. The inflation cuff may desirably contain polyurethane material and may desirably have a thickness between 5 and 30 microns.

Another aspect of the invention also addresses an airway ventilation device for insertion in an elongate body cavity. The airway ventilation device includes a tube having a distal end and a proximal end. The device also includes a toroidal shaped inflation cuff having an outer surface, an inner surface, a distal end, a medial portion, and a proximal end. The toroidal shaped inflation cuff is adapted to attach to the tube at least at one attachment zone on the tube. The at least one attachment zone, in comparison to the proximal end of the inflation cuff, is located at a distance further from the proximal end of the tube, and the at least one attachment zone, in comparison to the distal end of the inflation cuff, is located at a distance further from the distal end of the tube and wherein the outer surface is in communication with the trachea upon inflation.

Yet another aspect of the invention also addresses an airway ventilation device for insertion into an elongate body cavity. The airways ventilation device includes a tube comprising a distal end and a proximal end. The device also includes a toroidal shaped inflation cuff including an outer surface, an inner surface, a distal end, a medial portion, and a proximal end. The toroidal shaped inflation cuff is adapted to attach to the tube at least at one attachment zone on the tube. The at least one attachment zone, in comparison to the proximal end of the inflation cuff, is located at a distance further from the proximal end of the tube, and the at least one attachment zone, in comparison to the distal end of the inflation cuff, is located at a distance further from the distal end of the tube and wherein the outer surface is in communication with the trachea upon inflation. The at least one attachment zone also includes a slidable sleeve.

### BRIEF DESCRIPTION OF DRAWINGS

FIG. 1. is a perspective view of a prior art endotracheal tube having a non-toroidal inflation cuff and attachment zones located near the proximal and distal portions of the inflation cuff.

FIG. 2 is a perspective view of an endotracheal tube having a toroidal inflation cuff and two attachment zones.

FIG. 3 is a perspective view of an endotracheal tube having a toroidal inflation cuff and a single attachment zone

FIG. 4 is a perspective view of an endotracheal tube having a toroidal cuff and sliding attachment zones.

### DEFINITIONS

Toroid—The terms “toroid” or “toroidal” as used herein refers to a ring-shaped surface generated by rotating a circle around an axis that does not intersect the circle. It also refers to the donut shape common to such objects.
Residual Volume—The term residual volume refers to the amount by which the volume of the cuff in its unfolded state exceeds the volume of the cavity into which it is inserted.

### DETAILED DESCRIPTION

The airway ventilation device of the present invention provides for advanced endotracheal tube designs which are based on toroidal shaped subglottic inflation cuffs. The endotracheal tubes may include toroidal inflation cuffs which may be suitable for use with or without anti-microbial compositions and may include one or more recessed attachment zones located near the proximal and distal ends of the inflation cuff. Optionally, the attachment zone or zones may include a slidable sleeve.

The invention will be described with reference to the following description and figures which illustrate certain embodiments. It will be apparent to those skilled in the art that these embodiments do not represent the full scope of the invention which is broadly applicable in the form of variations and equivalents as may be embraced by the claims appended hereto. Furthermore, features described or illustrated as part of one embodiment may be used with another embodiment to yield still a further embodiment. It is intended that the scope of the claims extend to all such variations and embodiments.

In the interests of brevity and conciseness, any ranges of values set forth in this specification contemplate all values within the range and are to be construed as support for claims reciting any sub-ranges having endpoints which are whole number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

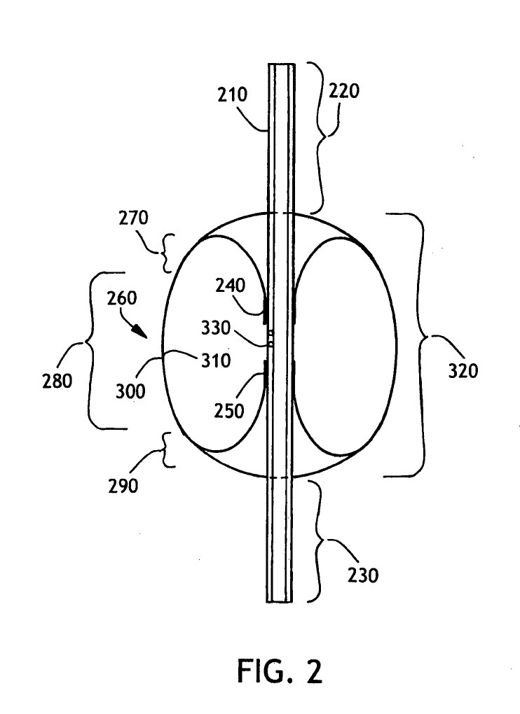

Referring to FIG. 2, an airway ventilation device for insertion into an elongate body cavity is provided. The apparatus includes an endotracheal tube 210 having a distal end 230, a proximal end 220, a medial portion 320 and at least two attachment zones including a first attachment zone 240 and a second attachment zone 250. The tube 210 may also include one or more openings 330 for introduction of fluids into an inflation cuff 260, the openings being connected to an inflation lumen, (not shown). These fluids, include gases and liquids.

The airway ventilation device, desirably an endotracheal tube, may include any flexible material which is compatible with the elongate body cavity into which it is inserted and which does not cause irritation of the cavity. These materials include, but are not limited to, latex, silicone, polyvinyl chloride, polyurethane or polytetrafluoroethylene.

The tube may be manufactured by any method known in the art of manufacturing tubing. A non-limiting example of a endotracheal manufacturing process suitable for this purpose is the coextrusion of two tubes (coextrusion being a process known and understood by those having skill in the manufacture of tubing), an outer tube and an inner tube. Additional, non-limiting examples include utilizing reinforcement inserts such as wire inside the shaft of the endotracheal tube, utilizing a spiral wound reinforcement, or utilizing a stabilizing mesh incorporated into the wall of the endotracheal tube.

Referring again to FIG. 2, the apparatus also include an inflation cuff 260 attached to the endotracheal tube 210 at the at least two attachment zones, 240 and 250. The inflation cuff includes an outer surface 300, an inner surface 310, a distal portion 290, a proximal portion 270, and a medial portion 280. The inflation cuff may be attached to the at least two attachment zones utilizing gluing, welding, or any other known method in the art for attaching an inflation cuff to a endotracheal tube. Desirably, the inflation cuff is toroidal in shape, unlike many prior art inflation cuffs which are non-toroidal in shape. Non-toroidal shapes include, but are not limited to, pear or bulb shaped inflation cuffs. The inflation cuff may be desirably pre-formed or may be alternatively formed by methods such as blow-molding, dip coating, or spin coating.

When the inflation cuff is pre-formed it is preformed to exhibit dimensions greater than the diameter of the elongate body cavity in which it is to be inserted, for example, the trachea. In this regard, it is preformed to exhibit dimensions from between about 1 to about 10 percent, more desirably between about 3 percent to about 7 percent beyond the diameter of the trachea. While these dimensions are variable, they may be readily determined by one of ordinary skill in the art utilizing conventional techniques.

The preformed inflation cuff is then attached to the endotracheal tube shaft at the at least two attachment zones, for example, a first attachment zone 240 and a second attachment zone 250. In this regard, the first attachment zone, in comparison to the proximal end of the inflation cuff, is located at a distance further from the proximal end of the tube. Additionally, the second attachment zone, in comparison to the distal end of the inflation cuff, is located at a distance further from the distal end of the tube and wherein the outer surface is in communication with the trachea upon inflation.

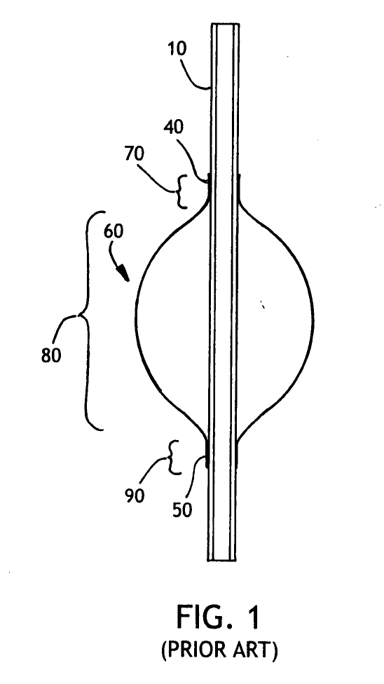

Thus, upon attachment of the inflation cuff to endotracheal tube, the first attachment zone and the second attachment zone will be located parallel to the medial portion 280 of the inflation cuff. Conversely, prior art (See FIG. 1) endotracheal tubes 10 having non-toroidal inflation cuffs 60 exhibit attachment zones 40, 50 which are located parallel to the proximal 70 and distal 90 portions of the inflation cuffs but are not located in a location parallel to the medial portion 80.

In this regard, when an inflation cuff is connected at attachment zones parallel to the proximal and distal portions of the inflation cuff such as in prior art inflation cuffs, transverse or longitudinal motion of the tube near the proximal and distal ends of the inflation cuff will dislodge a portion of the cuff from the tracheal wall. Conversely, endotracheal tubes having toroidal inflation cuffs which are moved in the same range of motion are less likely to be dislodged from the tracheal wall.

This stability of the toroidal inflation cuff is caused, in part, by the close proximity of the attachment zones to the center of the inflation cuff. For example, in a lever, the distance that an object is moved is proportional to the distance that the object is from the fulcrum (center). Because the attachment zones of the toroidal inflation cuff is relatively close to the center of the inflation cuff the inflation cuffs exhibit more stability and are much less likely to move than if the attachment zones were further away from the center of the inflation cuffs such as in prior art non-toroidal inflation cuffs.

Additionally, with toroidal inflation cuffs, there is more opportunity for annular rotation of the cuff without breaking the seal. In this regard, the toroidal inflation cuff acts like a watersnake toy (a toy having an elongated toroidal tube with a flexible plastic wall filled with water) whereby it may exhibit annular rotation while remaining in substantially stationary communication with the tracheal wall upon longitudinal or transverse movement of the endotracheal tube.

Advantageously, attachment zones located in the medial portion of toroidal shaped inflation cuffs allow the outer walls 300 of the inflation cuff to remain in substantially stationary communication with the tracheal cavity even when the endotracheal tube is moved in a longitudinal or transverse direction at a distance greater than the amount of longitudinal or transverse movement allowed by the residual volume of a non-toroidal shaped inflation cuff which remains in stationary communication with the trachea upon longitudinal or transverse movement. This helps prevent space from forming between the outer surface of the inflation cuff and the trachea, which blocks the entrance of pathogens into the respiratory system. This space forms because the residual volume in traditional inflation cuff allows leverage between the outer surface of the cuff and tracheal wall when the endotracheal tube is moved a substantial distance. Additionally, internally recessed attachment zones on toroidal inflation cuff allow for movement of the tube without pulling or pushing of the cuff longitudinally.

As a non-limiting example, it is contemplated that an adult Kimberly-Clark Microcuff endotracheal tube with a toroidal inflation cuff, the tube having a length of 35 cm, may be inflated to approximately 30 millibars inside the trachea and may be moved between 1 and 17.5 cm, more desirably between 7 and 14 cm in a longitudinal direction and maintain cuff seal in stationary communication with the tracheal cavity. Thus, the inflation cuff may desirably be maintained in stationary communication with the tracheal cavity when the tracheal tube is moved a distance equal to between 4 and 50 percent of the endotracheal tube length, more desirably between 20 and 40 percent of the tracheal tube length.

Similarly, as a non-limiting example, an adult endotracheal tube with a toroidal inflation cuff inflated to approximately 30 millibars, the tube having a diameter of 3 cm, may be moved in a transverse direction up to a distance equal to its diameter. In this regard, the endotracheal tube may be desirably moved in a transverse direction between 1.5 cm and 3 cm. Thus, the inflation cuff may be desirably maintained in stationary communication with the tracheal cavity when the tracheal tube is moved in a transverse direction a distance between 50 and 100 percent of the tube diameter, more desirably, between 65 and 80 percent of the tube diameter.

As an alternative to pre-forming, the inflation cuff may be constructed by any of the other various techniques well-known to those skilled in the art. For example without limitation, the polymer can be dip-coated on a mandrel that has a defined size and shape, desirably greater than the diameter of the tracheal cavity. When removed from the mandrel, the inflation cuff in its uninflated or collapsed state will assume two dimensions (length and width) of the mandrel without incurring any tensional force in the polymer.

The inflation cuff may also be formed by spin-coating in a hollow mold. When the mold is removed, as in the case of a dip-coated mandrel, the inflation cuff will assume dimensions that are the same as the interior dimensions of the hollow mold.

In addition, inflation cuffs may be formed by injection or blow molding. In this process, a pre-formed length of tubing made of the polymer is placed in a hollow mold having internal dimensions that reflect the desired dimensions of the inflation cuff to be formed. One end of the tube is sealed off and a working fluid is injected into the open end of the tube with sufficient force to cause the working fluid to expand the tubing until the wall of the tubing is in intimate contact with the inner surface of the mold. The polymer is then annealed, if desired, and cooled after which the mold is removed leaving a portion of the tubing as an inflation cuff.

The above are but a few methods of forming the inflation cuff. Others will be apparent to those skilled in the art. All such methods are within the scope of this invention.

Various materials may be used to form the inflation cuff. These materials include, but are not limited to, polyurethane (PU), low-density polyethylene (LDPE), polyvinyl chloride (PVC), polyamid (PA) or polyethylene teraphthalate (PETP). Additionally, copolymer admixtures for modifying the characteristics of the material may be used, for example a low density polyethylene and ethylene-vinylacetate copolymer (LDPE-EVA), or blends of the above mentioned materials (e.g. PU with PVC or PU with PA) would be considered suitable for forming the inflation cuff. Other materials would also be suitable so long as they exhibit properties enabling them to be processed into inflation cuffs having walls, desirably microthin walls on the order of about 5 to about 100 micrometers, more desirably into a range of between about 5 to about 50 micrometers, yet even more desirably between about 5 and about 20 micrometers, and yet even more desirably between about 5 and about 15 micrometers. It is also contemplated that the walls may have a thickness of less than about 5 micrometers. It if further contemplated that the thickness of the walls may vary. For example, the thickness of the portion of the inflation cuff adjacent the attachment zones may be up to 5 times thicker, desirably up to 3 times thicker than the thickness of other portions of the inflation cuff. Though the thickness of the walls of the inflation cuff may vary, it is desirable that the thickness of the inflation cuff material remain consistent throughout the inflation cuff.

The thickness of the walls of the inflation cuff may be measured by conventional measuring techniques. These techniques include the use of LITEMATIC which is a low-contact-force (0.01 N) thickness measuring device (LITEMATIC VL-50″ available from KABUSHIKI KAISHA MITSUTOYO, Japan).

It is also desirable, that upon inflation of the microthin inflation cuff, the inflation cuff will exhibit outward pressure towards the walls of the trachea, but will not exhibit upward pressure towards the mouth. Desirably, upon inflation inside the trachea, pressure inside the inflation cuff will be 30 millibars or less, desirably from about 10 to about 30 millibars, and an equal amount of pressure will be exerted upon the tracheal wall. It is also contemplated that the pressure could be less than 10 millibars for short periods of time during movement of the tube or during part of the breathing cycle.

These microthin walls are desirable because, as mentioned above, the diameter of the inflation cuff is greater than the diameter of the trachea. This allows the inflation cuff, upon inflation in the trachea to inhibit and/or arrest the free flow of secretions into the lungs. A detailed discussion of this phenomenon is found in U.S. Pat. No. 6,526,977 and U.S. Pat. No. 6,802,317 both of which are incorporated herein by reference in their entireties.

Additionally, it is desirable that the inflation cuff will demonstrate a low aspect ratio. The aspect ratio is determined by inflating the cuff to about 30 millibars and measuring the diameter and length which are both measured from the outer surface of the inflation cuff. In this regard, the aspect ratio (diameter:length) of the inflation cuff should be desirably 0.50 or less, more desirably 0.25 or less. As a non-limiting example, an inflation cuff having a diameter of 1 cm should desirably have a length of 2 cm or greater, more desirably 4 cm or greater.

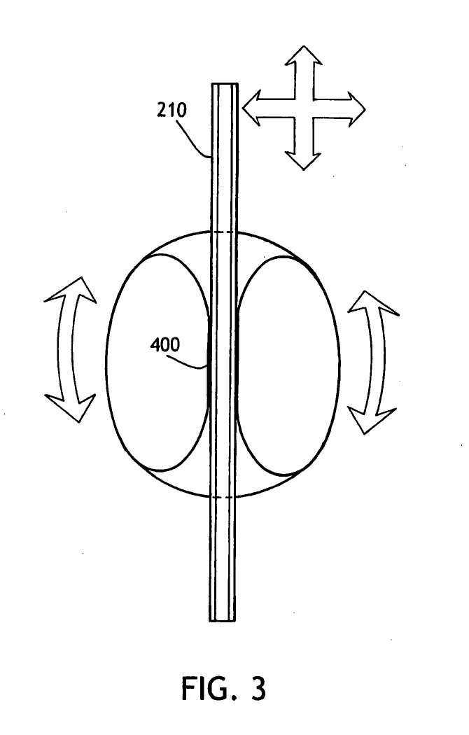

Turning to FIG. 3, the endotracheal tube may include a single recessed attachment zone 400. The single recessed attachment zone is formed when both first 240 and 250 are both recessed to a distance to where they coincide with each other and form a single attachment zone. For example, it is contemplated that when an inflation cuff having a single attachment zone is preformed, both ends of the inflation cuff will be brought together so that they are parallel and in communication with each other. The single attachment zone may then be glued to the endotracheal tube. It is also contemplated that upon longitudinal or transverse movement of an endotracheal tube having an inflation cuff with a single attachment zone, the inflation cuff will remain substantially in stationary communication with the trachea.

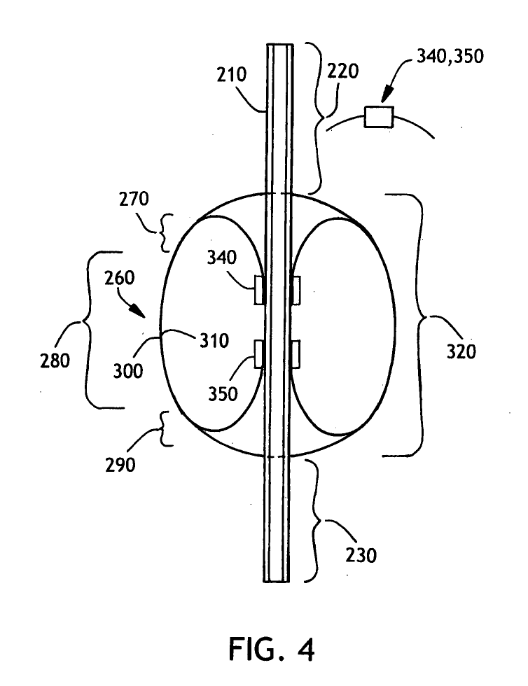

Turning to FIG. 4, the first attachment zone 340 and second attachment zone 350 may include sleeves which provide for a seal against secretions, but also allows for motion of the sleeve over the tube. The motion of the sleeve is similar to the motion of a slidable closure mechanism for a resealable bag.

This slidable sleeve my be formed from polyurethane (PU), low-density polyethylene (LDPE), polyvinyl chloride (PVC), polyamid (PA) or polyethylene teraphthalate (PETP) or any other material utilized to form the inflation cuff.

Optionally, the slidable sleeve may include antimicrobial agents within the sleeve. The anti-microbial agents provide extra protection in case any leverage between the cuff and the trachea is created form the movement of the inflation cuff over the tube.

These anti-microbial materials include, but are not limited to germicidal agents and biocides. Desirably, the use of anti-microbial materials will destroy or neutralize a broad spectrum of microorganisms including at a minimum Gram positive and Gram negative bacteria, including resistant strains thereof, for example methicillan-resistant Staphylococcus aureus (MRSA), vancomycin-resistant Enterococci (VRE) and penicillin-resistant Streptococcus pneumoniae (PRSP) strains. Desirably, they will destroy or neutralize all bacteria (Gram +, Gram − and acid fast strains) and yeasts such as Candida albicans.

These anti-microbial materials may be effective by themselves or may be combined to produce a synergistic effect that is non-additive of the individual components. These germicidal reagents may be further combined with processing aids and/or other ingredients that provide functional properties to the compositions.

Effective anti-microbial agents include, but are not limited to triclosan, chlorohexidine, charged Silver, Polyhexamethylene Biguanide, Chitosan glycolate, Octadecylaminodimethyl Trimethoxysilylpropyl Ammonium Chloride, N-Alkyl Polyglycoside, PG-Hydroxyethylcellulose Cocodimonium Chloride (Quaternary Ammonium CellulosicSalt), Xylitol, 2-hydroxy-1,2,3-propanetricarboxylic acid, Benzenecarboxylic acid, 2-hydroxybenzoic acid, Methane-carboxylic acid, 1,3-Propanedicarboxylic Acid, Iodine, Ethyl Hydroxyethyl cellulose, and Polyvinyl pyrrolidone.

It is contemplated that an adult Kimberly-Clark Microcuff endotracheal tube manufactured by Microcuff GmbH of Weinheim, Germany, and having a toroidal shaped inflation cuff may be placed in a graduated cylinder of inner diameter 21-25 mm (to simulate a trachea), the cylinder having a length of 60 cm. The cuffs may then be inflated with air to approximately 30 millibars to form a seal with the graduated cylinder. It is contemplated that 15 mL of deionized water containing a colored food dye may for poured over the inflated inflation cuff so that the water is resting upon the proximal end of the inflation cuff. It is also contemplated that the proximal end of the endotracheal tube may be pulled approximately 15 to 20 cm in an upward longitudinal direction towards the opening of the graduated cylinder. It is further contemplated that the 15 mL of colored water will remain on the proximal end of the inflation cuff and that the bottom of the graduated cylinder will remain substantially free of the colored water. It is also further contemplated that the inflation cuff will move less than 2 cm in an upward direction towards the opening of the graduated cylinder.

It should be noted that while the above specific examples show particular desired embodiments of the present invention, substitution of the specific constituents of those examples with materials as disclosed herein and as are known in the art may be made without departing from the scope of the present invention. Thus, while different aspects of the present invention have been shown and described, various modifications may be made without departing from the scope of the present invention, and all such modifications and equivalents are intended to be covered.

What is claimed is:

1. An airway ventilation device for insertion in an elongate body cavity, the device comprising:
a tube comprising a distal end, a proximal end, and at least two attachment zones; and
a toroidal shaped inflation cuff comprising an outer surface, an inner surface, a distal end, a medial portion, and a proximal end, wherein the toroidal shaped inflation cuff is adapted to attach to the tube at the at least two attachment zones, wherein a first attachment zone, in comparison to the proximal end of the inflation cuff, is located at a distance further from the proximal end of the tube, and wherein a second attachment zone, in comparison to the distal end of the inflation cuff, is located at a distance further from the distal end of the tube and wherein the outer surface is in communication with the trachea upon inflation.

2. The apparatus of claim 1, wherein the inflation cuff is inflated through one or more openings in the tube.

3. The apparatus of claim 2, wherein the inflation of the inflation cuff is accomplished by the introduction of a fluid.

4. The apparatus of claim 1 wherein the outer surface of the inflation cuff remains in stationary communication with the trachea when the tube is moved in a longitudinal or transverse direction, the movement of the tube being greater than the amount of longitudinal or transverse movement allowed by the residual volume of a non-toroidal shaped inflation cuff which remains in stationary communication with the trachea upon longitudinal or transverse movement.

5. The apparatus of claim 1, wherein the at least two attachment zones, upon inflation, are substantially aligned with the medial portion of the inflation cuff.

6. The apparatus of claim 1, wherein a portion of the inner surface of the inflation cuff is glued to the at least two attachment zones.

7. The apparatus of claim 1, wherein the inflation cuff comprises polyurethane material.

8. The apparatus of claim 1, wherein the thickness of the inflation cuff ranges from about 5 microns to about 30 microns.

9. An airway ventilation device for insertion in an elongate body cavity comprising:
a tube comprising a distal end, a proximal end, and at least one attachment zone; and
a toroidal shaped inflation cuff comprising an outer surface, an inner surface, a distal end, a medial portion, and a proximal end, wherein the toroidal shaped inflation cuff is adapted to attach to the tube at the at least one attachment zone, wherein the at least one attachment zone, in comparison to the proximal end of the inflation cuff, is located at a distance further from the proximal end of the tube, and wherein the at least one attachment zone, in comparison to the distal end of the inflation cuff, is located at a distance further from the distal end of the tube and wherein the outer surface is in communication with the trachea upon inflation.

10. The apparatus of claim 9, wherein the inflation cuff is inflated through one or more openings in the tube.

11. The apparatus of claim 10, wherein the inflation of the inflation cuff is accomplished by the introduction of a fluid.

12. The apparatus of claim 9 wherein the outer surface of the inflation cuff remains in stationary communication with the trachea when the tube is moved in a longitudinal or transverse direction, the movement of the tube being greater than the amount of longitudinal or transverse movement allowed by the residual volume of a non-toroidal shaped inflation cuff which remains in stationary communication with the trachea upon longitudinal or transverse movement.

13. The apparatus of claim 9, wherein the at least one attachment zone, upon inflation, is substantially aligned with the medial portion of the inflation cuff.

14. The apparatus of claim 9, wherein a portion of the inner surface of the inflation cuff is glued to the at least one attachment zone.

15. The apparatus of claim 9, wherein the inflation cuff comprises polyurethane material.

16. The apparatus of claim 9, wherein the thickness of the inflation cuff ranges from about 5 microns to about 30 microns.

17. An airway ventilation device for insertion in an elongate body cavity comprising:
a tube comprising a distal end, a proximal end, and at least one attachment zone; and
a toroidal shaped inflation cuff comprising an outer surface, an inner surface, a distal end, a medial portion, and a proximal end, wherein the toroidal shaped inflation cuff is adapted to attach to the tube at the at least one attachment zone, wherein the at least one attachment zone, in comparison to the proximal end of the inflation cuff, is located at a distance further from the proximal end of the tube, and wherein the at least one attachment zone, in comparison to the distal end of the inflation cuff, is located at a distance further from the distal end of the tube and wherein the outer surface is in communication with the trachea upon inflation, and wherein the at least one attachment zone comprises a slidable sleeve.

18. The apparatus of claim 17, wherein the slidable sleeve includes an antimicrobial agent.

19. The apparatus of claim 18, wherein the antimicrobial agent is triclosan, chlorohexidine, charged Silver, Polyhexamethylene Biguanide, Chitosan glycolate, Octadecylaminodimethyl Trimethoxysilylpropyl Ammonium Chloride, N-Alkyl Polyglycoside, PG-Hydroxyethylcellulose Cocodimonium Chloride (Quaternary Ammonium CellulosicSalt), Xylitol, 2-hydroxy-1,2,3-propanetricarboxylic acid, Benzenecarboxylic acid, 2-hydroxybenzoic acid, Methane-carboxylic acid, 1,3-Propanedicarboxylic Acid, Iodine, Ethyl Hydroxyethyl cellulose, or Polyvinyl pyrrolidone.
